(19) United States
(12) Patent Application Publication
(10) Pub. No.: US 2005/0191355 A1
(43) Pub. Date: Sep. 1, 2005

(54) Anti-microbial and antifungal fluid conduits and methods of manufacture thereof

(75) Inventor: Stephen W. Foss, (US)

Correspondence Address: PERKINS, SMITH & COHEN LLP, ONE BEACON STREET, 30TH FLOOR, BOSTON, MA, 02108, US

(73) Assignee: FOSS MANUFACTURING CO., INC., Hampton, NH (US)

(21) Appl. No.: 11/048,418

(22) Filed: Feb. 1, 2005

Related U.S. Application Data
(63) Continuation-in-part of application No. 10/406,720, filed on Apr. 2, 2003.
(63) Continuation-in-part of application No. 10/762,920, filed on Jan. 22, 2004.
Application No. 10/762,920 is a division of application No. 09/565,138, filed on May 5, 2000, now Pat. No. 6,723,428.
(60) Provisional application No. 60/136,261, filed on May 27, 1999.
(60) Provisional application No. 60/173,207, filed on Dec. 27, 1999.
(60) Provisional application No. 60/172,285, filed on Dec. 17, 1999.
(60) Provisional application No. 60/172,533, filed on Dec. 17, 1999.
(60) Provisional application No. 60/180,536, filed on Feb. 7, 2000.
(60) Provisional application No. 60/181,251, filed on Feb. 9, 2000.
(60) Provisional application No. 60/180,240, filed on Feb. 4, 2000.

(51) Int. Cl. A61K 9/14; A61M 31/00; A61K 33/38

(52) U.S. Cl. 424/486; 424/618; 604/500

(57) ABSTRACT

Antimicrobial/antifungal fluid conduits (are extruded, co-extruded, molded and/or otherwise thermoformed or thermoset), and films formed on non-thermoplastic conduit walls. One or more inorganic antimicrobial agents are selectively dispersed and concentrated near a surface at which antimicrobial/antifungal properties are desired. The agents resist wear from repeated fluid flows through embedding in a thin thermoplastic layer disposed upon the conduit wall. The fluid conduits preferably comprise high tenacity polymers (e.g. PET, PE, PP, ABS, PVC, Styrene, EVA) in at least one structurally supportive layer and the same or other thermoplastic or thermoset polymer in the thin inner layer including the antimicrobial agents.

### CROSS-REFERENCE TO RELATED APPLICATIONS

This application is a continuation-in-part of co-pending U.S. application Ser. No. 10/406,720 filed 2 Apr. 2003 and Ser. No. 10/762,920 filed 22 Jan. 2004, each of which is a divisional of (with the '720 application additionally being a continuation-in-part of) U.S. Pat. No. 6,723,428, which claims the priority of the following provisional applications: Ser. No. 60/136,261 filed 27 May 1999; Ser. No. 60/173,207 filed 27 Dec. 1999; Ser. No. 60/172,285 filed 17 Dec. 1999; Ser. No. 60/172,533 filed 17 Dec. 1999; Ser. No. 60/180,536 filed 7 Feb. 2000; Ser. No. 60/181,251 filed 9 Feb. 2000; and Ser. No. 60/180,240 filed 4 Feb. 2000. All of said applications are incorporated herein by reference as though set out at length herein.

### FIELD OF THE INVENTION

The present invention relates generally to products composed of synthetic, sheet materials and films having anti-microbial (and/or anti-fungal) properties which remain with the product after repeated exposures to turbulent or laminar fluid flows, and more particularly to thermoformable plastic piping, tubing and piping films exhibiting anti-microbial and anti-fungal characteristics.

### BACKGROUND OF THE INVENTION

There is a growing interest today in products which have anti-microbial and anti-fungal properties. Piping and tubing can provide rich environments for the formation of colonies of bacteria and fungi. A wide variety of industries, including food processors, commercial property management (e.g., water and HVAC), travel and tourism, sewage treatment facilities, airplane, ship, and train water systems, hospitals, and medical device and drug manufacturers expend vast amounts of money in environmental monitoring and maintenance of their fluid (e.g., water, steam, beverages, fuels, lubricants, blood, etc.) systems. Among their goals may be maintaining a particular fluid quality or purity, and/or preventing flow blockages caused by bacterial and/or fungal growth.

There are a number of additives and products on the market which claim to have these properties. However, many do not have such properties, or the properties do not survive the life of the product, or they have adverse environmental consequences.

Another disadvantage of some of the existing technologies is that the anti-microbial additives are organic and many organic materials either act as antibiotics and the bacteria “learns” to go around the compound, or many of them give off dioxins in use. Examples of some organic types of anti-microbial agents, are U.S. Pat. Nos. 5,408,022 and 5,494,987 (an anti-microbial polymerizable composition containing an ethylenically unsaturated monomer, a specific one-, di- or tri-functional anti-microbial monomer and a polymerization initiator which can yield an unreleasable anti-microbial polymer from which the anti-microbial component is not released), U.S. Pat. No. 5,709,870 (a silver containing anti-microbial agent which comprises carboxymethylcellulose, a crosslinked compound, containing silver in the amount of 0.01 to 1% by weight and having a degree of substitution of carboxymethyl group of not less than 0.4 and the anti-microbial agent being a silver salt of carboxymethylcellulose, which is insoluble to water), U.S. Pat. No. 5,783,570 (an organic solvent-soluble mucopolysaccharide consisting of an ionic complex of at least one mucopolysaccharide and a quaternary phosphonium, an antibacterial antithrombogenic composition comprising organic solvent-soluble mucopolysaccharide and an organic polymer material, an antibacterial antithrombogenic composition comprising organic solvent-soluble mucopolysaccharide and an inorganic antibacterial agent, and to a medical material comprising organic solvent-soluble mucopolysaccharide).

Examples of some inorganic types of anti-microbial agents are:

Japanese Patent No. 1246204 (1988) which discloses an anti-microbial thermoplastic article with copper a compound added to the melted polymer just before extruding, in which the anti-microbial material is said to be resistant to washing.

U.S. Pat. No. 5,180,585 which discloses an antimicrobial with a first coating providing the antimicrobial properties and a second coating as a protective layer. A metal having antimicrobial properties is used including silver which is coated with a secondary protective layer.

The use of anti-microbial agents in connection with thermoplastic material is known from U.S. Pat. No. 4,624,679 (1986). This patent is concerned with the degradation of anti-microbial agents during processing. This patent states that thermoplastic compounds which are candidates for treatment with anti-microbial agents include material such as polyamides (nylon 6 or 6,6), polyvinyl, polyolefins, polyurethanes, polyethylene terephthalate, styrene-butadiene rubbers.

Many antimicrobial additives are applied topically to surfaces at which microbial control is desired, and tend to wash or wear off over time and become ineffective. If utilized in a closed water recirculation systems, for example, this would place a greater burden on the associated filtration equipment.

Thus, there exists a need to develop durable inorganic or metal antimicrobial agent containing piping and tubing products that do not permit the development of resistant bacterial strains. There also exists a need for the anti-microbial agents employed to resist being abraded or washed away by constant or intermittent turbulent flows and elevated fluid pressures and/or temperatures, thus maintaining their potency as an integral part of the piping or tubing into which they are incorporated.

PETG, as the abbreviation is used herein, means an amorphous polyester of terephthalic acid and a mixture of predominately ethylene glycol and a lesser amount of 1,4-cyclohexanedimethanol. It is known that PETG can be used in polycarbonate blends to improve impact strength, transparency, processability, solvent resistance and environmental stress cracking resistance.

Udipi discloses in U.S. Pat. Nos. 5,104,934 and 5,187,228 that polymer blends consisting essentially of PC, PETG and a graft rubber composition, can be useful as thermoplastic injection molding resins.

Chen et al. in U.S. Pat. No. 5,106,897 discloses a method for improving the low temperature impact strength of a thermoplastic polyblend of PETG and SAN with no adverse effect on the polyblends clarity. The polyblends are useful in a wide variety of applications including low temperature applications.

Billovits et al. in U.S. Pat. No. 5,134,201 discloses that miscible blends of a thermoplastic methylol polyester and a linear, saturated polyester or co-polyester of aromatic dicarboxylic acid, such as PETG and PET, have improved clarity and exhibit an enhanced barrier to oxygen relative to PET and PETG.

Batdorf in U.S. Pat. No. 5,268,203 discloses a method of thermoforming thermoplastic substrates wherein an integral coating is formed on the thermoplastic substrate that is resistant to removal of the coating. The coating composition employs, in a solvent base, a pigment and a thermoplastic material compatible with the to-be-coated thermoplastic substrate. The thermoplastic material, in cooperation with the pigment, solvent and other components of the coating composition, are, after coating on the thermoplastic substrate, heated to a thermoforming temperature and the thermoplastic material is intimately fused to the thermoplastic substrate surface.

Ogoe et al. in U.S. Pat. No. 5,525,651 disclose that a blend of polycarbonate and chlorinated polyethylene has a desirable balance of impact and ignition resistance properties, and useful in the production of films, fibers, extruded sheets, multi-layer laminates, and the like.

Hanes in U.S. Pat. No. 5,756,578 discloses that a polymer blend comprising a monovinylarene/conjugated diene black copolymer, an amorphous poly(ethylene terephthalate), e.g. PETG, and a crystalline poly(ethylene terephthalate), e.g. PET, has a combination of good clarity, stiffness and toughness.

Ellison in U.S. Pat. No. 5,985,079 discloses a flexible composite surfacing film for providing a substrate with desired surface characteristics and a method for producing this film. The film comprises a flexible temporary carrier film and a flexible transparent outer polymer clear coat layer releasably bonded to the temporary carrier film. A pigment base coat layer is adhered to the outer clear coat layer and is visible there through, and a thermo-formable backing layer is adhered to the pigmented base coat layer. The film is produced by extruding a molten transparent thermoplastic polymer and applying the polymer to a flexible temporary carrier thereby forming a continuous thin transparent film. The formed composite may be heated while the transparent thermoplastic polymer film is bonded to the flexible temporary carrier to evaporate the volatile liquid vehicle and form a pigment polymer layer. The heating step also molecularly relaxes the underlying film of transparent thermoplastic polymer to relieve any molecular orientation caused by the extrusion. Ellison also mentions that it is desirable to form the flexible temporary carrier from a material that can withstand the molten temperature of the transparent thermoplastic polymer. The preferred flexible temporary carriers used in his invention are PET and PETG.

Sheet materials for various uses are vulnerable to the seeding of bacteria and fungi from various sources, thus providing hospitable sites for their uninhibited growth. The latter is especially true since, depending upon the end use, they often are used in environments where there is great exposure to microbes and fungi. One example is cafeteria trays. Thus, these materials would benefit from having antibacterial and anti-fungal agents incorporated onto them and/or into them. However, most prior art approaches of providing sheet materials with anti-microbial or anti-fungal agents have limited effect.

A variety of patents relate to anti-microbial materials being added to materials. For example, U.S. Pat. No. 3,959,556 (1976) relates to synthetic fibers that incorporate an anti-microbial agent. U.S. Pat. No. 4,624,679 (1986), mentioned above, uses anti-microbial agents in connection with thermoplastic materials. These materials are formed by mixing polyamide resins, anti-microbial agents, and an antioxidant for reducing the degradation of the anti-microbial agent at the high temperatures necessary for processing.

Several other patents describe anti-microbial materials in which the anti-microbial agent is resistant to being washed away. U.S. Pat. No. 4,919,998 (1990) discloses an anti-microbial material that retains its desirable properties after repeated washings.

However, these materials have inherent commercial disadvantages. While the anti-microbial agents incorporated into them do show some resistance to repeated washings, these agents do leach out of the materials, primarily because they are not physically incorporated into them. In fact, in many cases, the anti-microbial agents are only loosely bound into the material and are relatively easily washed away or naturally abraded away over time. On the other hand if the agents are buried too deeply in the material or homogeneously distributed they will not contact microbes at all and the economics of usage will be adversely affected.

U.S. Pat. No. 4,923,914 for a Surface-Segregatable, Melt-Extrudable Thermoplastic Composition discloses forming a fiber or film of polymer and an additive in which the additive concentration is greater at the surface for example when surfactants are added to polymers to impart a special property thereto such as a hydrophilic character to the surface, if the additive is compatible with the polymer there is a uniform concentration of the additive throughout the polymer. In the past such webs have been bloomed to bring the surfactant to the surface. But the surfactant is incompatible at melt-extrusion temperatures. The patentee describes a process for overcoming this problem.

However, the process described has not been very usable with anti-microbial agents. For example, see U.S. Pat. No. 5,280,167 which describes the '914 patent discussed above and states that previous attempts to apply the teachings thereof to the preparation of non-woven webs having anti-microbial activity were not successful. This '167 patent provides for delayed anti-microbial activity in order to delay the segregation characteristic of the '914 patent from occurring. The additive which is used is a siloxane quaternary ammonium salt, an organic material.

While these anti-microbial agents are designed to prevent the development of resistant bacterial strains, the use of metal-containing materials presents the added difficulty of being able to successfully disperse the anti-microbial agents throughout the material. Since these metal-containing compounds exists as fairly large size particles (10 microns and greater), the ability to evenly mix or distribute them is limited. In addition, because of this size problem, these substances must necessarily be applied to the surfaces of materials instead of being incorporated into them. The latter causes the additional disadvantage of making the applied anti-microbial agents relatively labile to washings or abrasion.

Thus, there still exists a need to develop antimicrobial/antifungal thermoformable sheet materials and films for various uses that do not cause the development of resistant bacterial strains.

### SUMMARY OF THE INVENTION

It is an object of the present invention to provide antimicrobial properties in a lasting, efficacious and economical manner to fluid transporting components. As the term is used herein, “antimicrobial” properties refers collectively to antimicrobial, anti-fungal and/or anti-mold properties. Also, the term “conduit”, as used herein, is meant to encompass fluid transporting components including, but not limited to, piping, tubing, canals, channels, ducts, flumes, gutters, sewers, spouts, and troughs, any of which may be thermoformed or thermoset, and to any of which a thermoformed or thermoset film may be applied.

It is also an object of the present invention to utilize inorganic anti-microbial additives such as, for example, copper, zinc, tin, silver or any inorganic antimicrobial agent, in the form of particles that may or may not be carried in carriers such as zeolites, zirconium phosphate and/or dissolvable glass.

It is another object of the present invention to provide fluid conduits or films for application thereto in which the anti-microbial agent is present in or applied to only predetermined regions where the antimicrobial effect is desired, thereby reducing the amount of antimicrobial agent needed and thus lower the production costs of such products.

It is a further object of the present invention to impart properties to the conduits, beyond the antimicrobial properties, through the use of additives such as: color pigments to make the conduits color-fast; UV additives to resist fading and degradation in conduits exposed to significant UV light; hydrophilic or hydrophobic additives; flame retarders; and/or anti-stain additives.

It is a principal object of the present invention to satisfy the needs discussed above in a manner consistent with industry specifications, overall durability, and cost-effectiveness.

The foregoing objects are met by antimicrobial fluid conduits, such as piping or tubing, having a wall upon which is thermoformed or thermoset a plastic polymer layer in which antimicrobial particles are dispersed in an efficacious concentration that is constrained by the thickness (or thinness) of the plastic polymer layer to a region at the surface of the layer, such that an antimicrobial effect is obtained at that surface. The choice of particle size of the agent carriers depends upon the thickness of the plastic layer or film. In order to obtain the best combination of surface area with anchoring in the film or plastic layer, the thickness of such film or layer should be in the range of 1.73 to 2.5 times the nominal size of the (typically cubic) antimicrobial particle. Relating the thickness to the antimicrobial particle size ensures that a substantial portion of the antimicrobial particles will have a portion exposed through the surface of the plastic layer. For example, a very thin film/co-extruded layer of 3 μm would be best served with a zeolite having a nominal particle size of 1-2 μm, which would have a maximum dimension of 2×1.73 or approximately 3.5 μm.

The antimicrobial agents are, thus, optimally arranged at the surface of the thin plastic layer such that portions of the agents are exposed to the fluid being transported and in which microbial control is sought. The concentration of antimicrobial additive in the plastic layer is a function of the anticipated microbial load in the fluid being transported, or, in the case of external pipe covers, the environment to which the pipe is to be subjected.

The antimicrobial layer may be formed on either or both “inner” or “outer” pipe or tube wall surfaces, and may comprise a plastic layer co-extruded with the conduit wall, or alternatively may comprise a thermoformed or thermoset film later applied to a conduit such as a metallic (e.g., steel, aluminum, copper) pipe. Either or both the conduit wall and plastic layer may be composed of one or more of the following: polyethylene (PE), polypropylene (PP), polyethylene terephthalate (PET), polyamide (Nylon), aramid, acrylic, styrene or any thermoformable or thermoset polymer. The wall and plastic layer may additionally be formed by injection molding, rotational molding, or any process that allows shaping into the desired conduit shape.

The concentration of the anti-microbial agents can be varied as a gradient using mixing strategies. The concentration of anti-microbial agents within or on the surface of the film can also be varied regionally using materials containing varying amounts of anti-microbial agents in conjunction with both natural and synthetic materials having different amounts of anti-microbial agents or even no added anti-microbial agents. The co-extruded or molded conduits and/or films may be cut to a desired length or may be infinite in length, and may be formed in diameters ranging anywhere between 0.1 inches to 18 feet.

### BRIEF DESCRIPTION OF THE DRAWING

Other objects, features and advantages will be apparent from the following detailed description of preferred embodiments taken in conjunction with the accompanying drawings in which:

FIGS. 1 and 2 are cross-sectional views of cylindrical piping having an antimicrobial outer layer and an inner wall;

FIG. 3 is a sectional view through the exit of an extruder showing the formation of co-extruded conduit wall and antimicrobial layer in accordance with a piping or tubing embodiment of the present invention;

FIG. 4 is a schematic view of a feed hopper, screw and extruder;

FIG. 5 is a side view of a sheet material having an anti-microbial film layer co-extruded thereon thermoformable into a fluid conduit in accordance with the present invention;

FIG. 6 is a side view of a sheet material having two anti-microbial films extruded thereon, one on each side;

FIG. 7 is a side view of a further arrangement in which a double sheet material is complete surrounded by an anti-microbial film;

FIG. 8 is a side view of a shaped sheet material having two anti-microbial films extruded thereon;

FIG. 9 is a partial sectional view of apparatus for making a multi-layer co-extruded sheet thermoformable into a conduit in accordance with the present invention;

FIG. 10 is a sectional view through the apparatus shown in FIG. 9; and

FIG. 11 is an isometric view of apparatus for making a side-by-side co-extruded sheet.

### DESCRIPTION OF PREFERRED EMBODIMENTS OF THE INVENTION

The description below provides several particular methods for manufacturing antimicrobial fluid conduit profiles, none of which are intended to limit the scope of the invention. As noted, fluid conduits in accordance with the invention may have closed profiles such as, for example, in solid or hollow piping, tubing (e.g., and films for same) embodiments, or could have open profiles such as in gutter or trough embodiments. All embodiments, however, share a characteristic wherein antimicrobial particles are constrained to regions at or near the interior and/or exterior surfaces of the conduit where an antimicrobial effect is desired. By selecting a film or outermost layer thickness that is approximately twice (between roughly 1.73 and 2.5 times) the nominal size of the antimicrobial particles utilized, the maximum efficacy is achieved with a minimal use of agents and therefore reduced cost. In a preferred embodiment, the conduit layers co-extruded, but any thermoforming (e.g., molding) or thermosetting method may be employed. In the descriptions below, terms such as “sheath”, “lining” and “layer” are but alternative expressions referring to the antimicrobial term plastic layer/film. The conduit wall itself is also occasionally referred to as a layer because the wall is preferably also composed of a thermoplastic polymer allowing formation simultaneous with the antimicrobial layer, but in certain embodiments may be composed of metals (e.g., aluminum, copper, steel) or even concrete.

Antimicrobial layers or films consistent with the invention may be formed as outer sheaths or inner linings to otherwise non-microbial conduit profiles. The sheaths and/or linings are preferably quite thin but capable of withstanding repeated turbulent and/or laminar fluid flows. The antimicrobial particles may be dispersed in a polyethylene (PE), PET, PP, polybutylene terephthalate (PBT) or any thermoplastic carrier, but could also be added directly to a thermoplastic melt without an intermediate carrier in forming the antimicrobial layer or film. The agents are bound within the layer and are thereby prevented from washing off over time and remain effective, especially when the plastic layer to antimicrobial agent particle size ratio is in the range noted above. Additional additives that can be incorporated into the thermoplastic melt prior to thermoforming of the plastic layer include one or more UV stabilizers, fire retardants, pigments, and/or hydrophilic or hydrophobic additives.

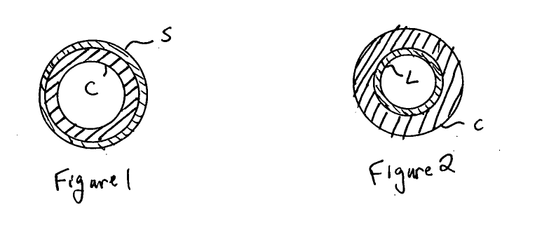

FIGS. 1 and 2 illustrate piping-type profiles of the conduit having an outer antimicrobial sheath S and inner antimicrobial lining L, respectively. Two polymeric layers, one providing mechanical strength C (and referred to herein as the conduit's “wall”) and the other antimicrobial properties can be co-extruded in a closed, cylindrical (or other) profile to produce the piping or tubing. The conduit wall may be composed of PET or another high tenacity polymer. Poly 1,4 cyclohexylene dimethylene terephthalate (PCT) or another hydrolysis resistant polymer may be used to form the antimicrobial plastic layer (i.e., the sheath and/or lining) for long-term wrinkle resistance and biding strength. In the production of tubing, the modulus (the area under the curve in a stress/strain curve) of the supporting layer can be varied as desired for the tubing's end use.

The respective layers of the fluid conduit may be formed from pellets of the same or different polymers, one having interspersed therein an effective concentration (e.g., 0.05% to 8%) of antimicrobial particles, possibly in a carrier such as zeolites, zirconium phosphate or dissolvable glass. It has been shown that a synergistic anti-fungal property can be imparted to the thin layer through the use of combinations of antimicrobial agents, preferably combinations of copper and silver particles range from about 10% Ag: 90% Cu to about 89% Ag: 10% Cu and about 1% Zn. Since abrasion at the surface(s) may occur, some of the agents dispersed just below the surface when the antimicrobial layer is made become available at the surface, later in the life of the product. Particles of zeolite of silver may be produced in roughly cubic shapes from sub-micron to larger sizes. As an example, a 1 micron particle will have a diagonal dimension of about 1.7 micron. Therefore, the smallest thickness of the sheath or lining should be about 2 microns (in a range of 1.73 to 2.5 times the nominal size of the particle.) The dimensions of the thicker supporting layer are limited only by the particular application.

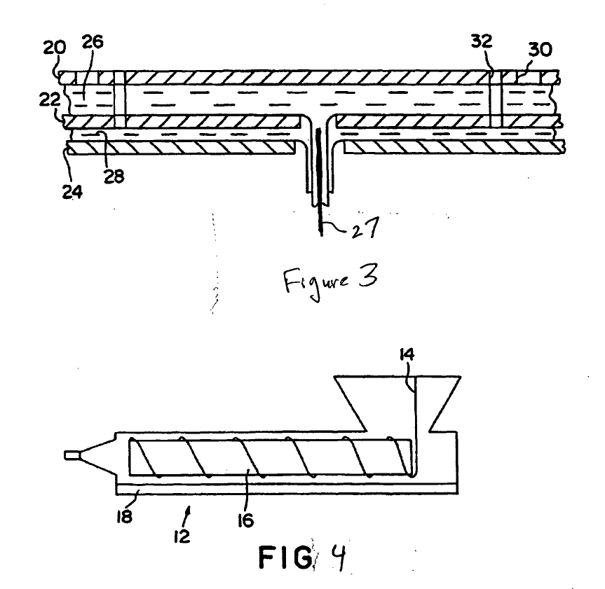

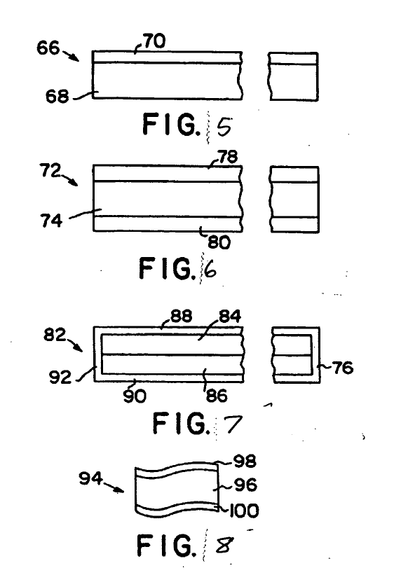

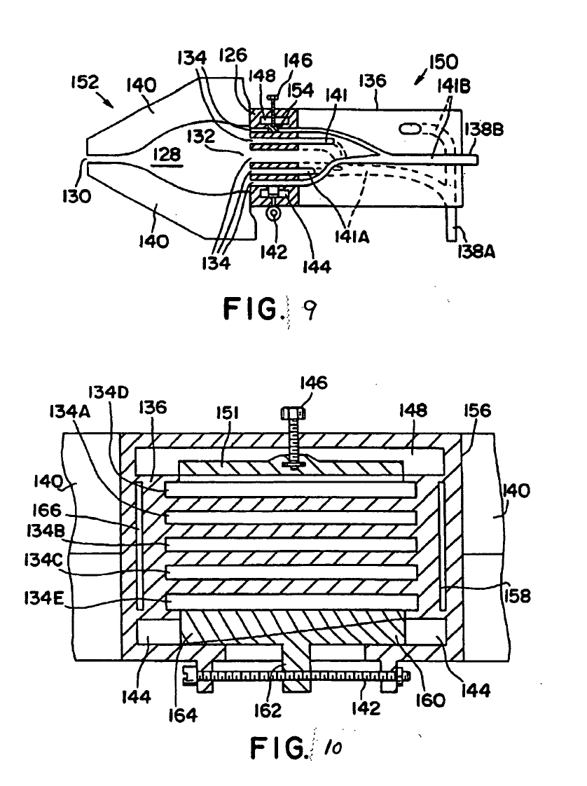

FIGS. 3 and 4 show equipment for co-extruding hollow piping or tubing profiles including a supporting conduit wall and an outer sheath/layer having embedded therein one or more antimicrobial particle types. The extruder 12 is shown diagrammatically in FIG. 4 having a feed hopper 14, an extruder screw section 16 for feeding melted material to the delivery end, and a heating chamber 18 which surrounds the bottom of the feed hopper as well as the total length of the extruder screw section 16 for melting the pellets which are fed into the hopper and maintaining the polymers in melted condition for being extruding through the extruding openings which act as nozzles. In an alternative to the use of pellets, it is possible to make the piping and/or tubing using direct polymer streams from continuous reactors feeding to the melt pumps.

Although not shown, there are two extruders, one which has a feed hopper for forming the antimicrobial sheath layer and another with a hopper for forming the supporting hollow wall of the piping profile. Piping having a PET wall and a PETG sheath containing one or more antimicrobial agents can be formed as follows: PETG pellets are placed into the first extruder and PET pellets are placed into the second extruder; both are heated sufficiently so that the extruders cause the melts to flow to a single extrusion nozzle in which the PET is made into the hollow wall and the PETG is made into the sheath. The nozzle end of the extruder is shown in cross section in FIG. 3 which includes three sheets of metal 20, 22 and 24 to form two chambers 26 and 28. The melted polymer is fed into the extruder nozzle from the top. There are a plurality of two types of holes, one type being 30 and which feeds into chamber 26 and along hollow die 27 to form the hollow wall of the piping profile, and the other type being 32 which feeds into chamber 28 to form the sheath of the fluid conduit. Those of skill in the art will readily appreciate that extruder configurations can be devised wherein thin antimicrobial layers are co-extruded on either or both the inner and outer surfaces of the conduit wall.

A variety of mono- or multi-layered profiles useful in fluid transmission systems, such as duct work, sanitary and water piping, gasket materials, medical tubing and the like can be formed from extrusion and/or molding (e.g., injection) processes. In order to produce rigid conduits, high tenacity polymers (e.g. PET, PP, ABS) may be utilized as in the conduit wall formation. Similar design controls regarding the thickness of the antimicrobial layers relative to the antimicrobial particle sizes, however, are applicable to the production of antimicrobial films for conduit walls not composed of thermoplastics. Such films may be composed of basically any thermoplastic resin, such as PE, PP, PET, EVA, PS, Polyamide (nylon), Acrylic, PVC, etc., and mixtures thereof, and may additionally be mixed with up to 15% of one or more low temperature polymers such as PETG, Polycaprolactone, EVA, and the like or hydrolysis resistance polymers (e.g. PCT). The presences and/or concentration of antimicrobial agents in the film may be controlled by properly timing the addition of the agents during film or profile production.

FIG. 5 shows a portion of an antimicrobial multi-layer sheet that can be shaped into a fluid conduit profile in accordance with the present invention. The multi-layer sheet 66 has a main, thicker support layer 68 (that can comprise the conduit wall) and a thin plastic layer 70 of thermoplastic material in which are interspersed particles of anti-microbial agent(s) constrained to the surface or just below the surface of the layer. In this way the anti-microbial particles are anchored into the plastic layer 70 and therefore remain there for the life of the conduit made from the sheet material and provide anti-microbial properties for the entire time.

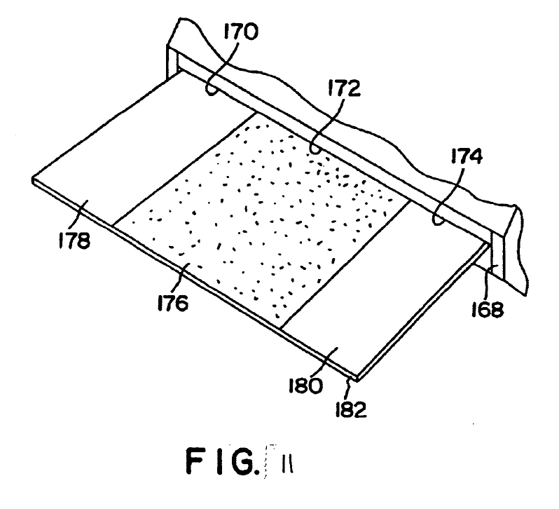

Another type of laminate sheet construction that may be used to form fluid conduit profiles is shown in FIG. 6. In this arrangement, the laminate sheet 72 has a main support layer 74 (to form the conduit wall) and each surface (“inner” and “outer”) of the wall layer have antimicrobial layers 78 and 80, respectively. One or both of the layers 78 and 80 include dispersed anti-microbial agents. Layer 74 is a wide sheet of material which may be extruded of thermoplastic material. It can be a rigid material or a flexible material depending upon the end use. The second and third layers are adhered to it by suitable means known in the art or they may be co-extruded as described below in connection with FIGS. 9-11. All three layers may be co-extruded simultaneously so as to bond them together immediately after extrusion and while the layers are still hot and prior to quenching, during which time they may also may be deformed into the desired fluid conduit profile.

It is possible to form the three layer laminate sheet 72 which includes the support (“wall”) layer 74 of at least 10 microns contemporaneously with a second sheet 78 to form a two-layer sheet, the second sheet being 4 microns in thickness and being supported by the first layer. The extruding of both layers is done at the same time and the second sheet 78 is joined to the first sheet 74 before the quenching is complete. If desired, a third sheet 80 similar to the second one, 78, can be made at the same time. The second and third sheets may have one or more anti-microbial agents of the type discussed herein mixed with the thermoplastic material so that the three layer sheet has a thin top layer and a thin bottom layer possessing anti-microbial properties.

FIG. 7 shows a laminate film 82 comprised of a first inner layer 84 and a second inner layer 86 having two surface layers 88 and 90 and, if desired, edge layers 92 and 76. FIG. 8 shows a laminate film 94 having a curved profile and including a center support layer 96 and two surface layers 98 and 100.

Forming Co-Extruded Laminate Sheets

With reference to FIGS. 9 and 10, a suitable die has a funnel-shaped expansion chamber 128 terminating in a slotted die outlet 128 defined by a pair of spaced die lips. The die has a shallow chamber entrance section 132. The feed block 126 comprises a plurality of slotted layer distribution passages 134 in the form of mutually spaced apart slots or openings lying substantially parallel to slotted die outlet 128. The passages extend from an inlet side to an outlet side of the feed block 126.

The feed block further comprises end encapsulation slots 166 and 158 extending between inlet and outlet sides without intersecting passages 134 and lying substantially perpendicular thereto. Otherwise, slots 166 and 158 may extend along planes converging together from the inlet side to the outlet side. The feed block assembly 152 includes a frame 136 connected to the upstream end of the die in some suitable manner and defining a chamber (not shown) open on opposite sides to facilitate removal and replacement of feed block 126 with an interchangeable feed block designed to accommodate specific resin viscosities, selected polymer matchups, layer thickness changes, layer geometry, etc.

Frame 136 includes various connectors 138A and 138B to which extruders (not shown) of polymer melts are connected, and to which feed channels or feed lines (also not shown) are likewise connected for feeding the melts to slots 134A-134E, 166 and 158, or to selected ones thereof. The feed block may be connected in some suitable manner to frame 136 or may be unconnected thereto.

Apparatus generally designated 152 is illustrated in FIGS. 9 and 10 as comprising a slit die 140 of mating die halves. A feed block assembly, generally designated 150, is totally integrated into the die as it is inserted within a die cavity 156 open at the upstream end of the die and at opposing sides of the die, shown in FIG. 10. Feed block assembly 150 comprises feed block 126, connectors 138A and 138B and melt feed lines 141A and 141B, respectively, extending from the connector 138A for feeding plastic melt from the extruder to the slotted passages 134A, 134B and 134C, and from the connector 138B for feeding plastic melts from the extruder to the slotted passages 134D and 134E. When one or more an antimicrobial agents or other additives are to be provided in the thinner outer sides of the laminate sheet, the agents or additives are added into the melt which is then extruded and fed to feed line 141B and connector 138B to extruding slots 134D and 134E. In the event the edges of the laminated sheet is to differ from the material fed into feed lines 141A and 141B, a third feed line (not shown) can be connected to slotted passages 166 and 158 of the feed block. If the edges are not to be different the slotted passages 166 and 158 are not or may be omitted from the construction of feed block 126. Thus, the entire feed block assembly 150 can be removed from cavity 156 and replaced by another feed block assembly for a new production cycle.

Feed block 126 of apparatus 152 can be provided with externally accessible means to control the melt streams of polymer melt passing through the outermost slots 134D and 134E for adjusting the distribution of the outer or skin layers of the skin laminate to be formed. Such control means may be in the form of a restrictor bar 154 extending transversely to the direction of flow of melt through the passages for controlling the width and/or shape of the outermost passage upon manual manipulation of an adjustment screw 146. The restrictor bar may be located in a side cavity 148 of the feed block.

Otherwise, the layer control means may be in the form of a driven wedge 164 mating with a drive wedge 160 connected to a screw drive 142 via flange 162, as more clearly shown in FIG. 10. The wedges may be housed in a suitable side cavity 144, and a turning of screw drive 142 shifts wedge 160 along the screw drive and causes the driven wedge to be shifted transversely relative to the melt flow through the feed block for controlling the distribution of the antimicrobial, fluid-contacting (or “skin”) layer flowing through the outer-most passage of the feed block.

Restrictor bar 154 can be utilized on both sides of the feed block, and the wedge arrangement can likewise be utilized on both sides. Restrictor bar 154 and wedge 164 can have flat melt flow engaging surfaces, or these surfaces can be concavely or convexly shaped or otherwise contoured to control the layer distribution of the skin layers by modifying the outer slots to accommodate differences in melt viscosities, etc.

With this arrangement one or both outer layers may have anti-microbial agents dispersed therein. In one example, a three-layer configuration can be made to have a center layer of 10 microns and the outer layers may be 4 microns, with a particle size of about 1.5-2 microns. If zeolite of silver particles are used and made this size then substantially every particle of zeolite will have at least a portion exposed by projecting through the outer surface of the layer in which it is embedded.

FIG. 11 shows a die 168 having a single extrusion slot with three portions, 170, 172 and 174. The sheet which is extruded thereby is shown having a center section 176 and two edge portions 178 and 180. The width of the center portion 176 is the same as the widths of the edge portions together. During the extrusion process, die slot portion 170 produces edge portion 178, die slot portion 172 produces center portion 176 and die slot portion 174 produces edge portion 180. The stippling indicates that an anti-microbial and/or an anti-fungal agent has been incorporated into the center portion of the extruded sheet. The extruded laminate film is shown having a thickness 182 which is the same throughout, although portions could be of different thickness if this is desired.

Although most of the examples shown illustrate relatively flat sheets being produce, non-flat fluid conduit profiles can be produced by the described process. For the piping and tubing applications, circular profiles are typically appropriate to serve as outer sheaths and/or inner linings, whereas simple rectangular or more complex shapes may be appropriate to serve as sheaths or linings for other fluid transport and/or storage system components. The forms taken are driven by the end uses. Also, as in the co-extrusion processes described above, other suitable non-antimicrobial additives may be added to provide other properties to the films and co-extruded profiles.

Higher loading of the anti-microbial agents (up to 5 times), or combinations of antimicrobials, may be necessary to more effectively act against fungi. This higher loading may be achieved by using various zeolites followed by heating the film polymer, e.g. PET, to between 180 and 228 degrees Fahrenheit in hot water which allows further metal loading or ion exchange to replace resident metal ions with another ion or mixture of ions. In addition, this would allow the zeolite at or near the surface of the film to be preferentially loaded with the metal ion or mixtures thereof that has the desired biological effect. These methods are particularly useful in reducing costs when expensive metal ions, such as silver, are used in these processes. Also, by adding certain metals, e.g. silver, at this point in the process and not having it present during the high temperature film extrusion process, any yellowing or discoloration due to oxidation of the metal ion or its exposure to sulfur and halogens would be greatly reduced.

Effective antibacterial protection is preferably achieved by incorporating between 0.1 and 20 percent by weight of the agents into the laminate films and/or profiles. Alternatively, the anti-microbial agent concentration can be reduced to between 0.2 and 6.0 percent in multi-layer sheets in which the anti-microbial agent is only mixed into the outer layer(s) of the laminate films and profiles.

It is similarly possible to vary the concentrations of agents in different portions of the films and profiles by agent adjustments during mixture with the film-forming polymers. The amount of anti-microbial agents present in the film can be varied either lengthwise or in cross-section.

In addition, the films and profiles can be made either hydrophilic or hydrophobic as desired by mixing other agents into the polymeric extrusion materials or applying them to the film surface. By modifying the wetability characteristics of the films or profiles, they can be made more useful for various applications. For example, hydrophilic films are effective in applications in which one wants the antimicrobial laminate to more easily absorb water, such as when the material is designed to be used in humid conditions. Alternatively, hydrophobic films are effective in applications in which one wants to avoid the absorption of such solutions.

The anti-microbial agents can also be added to low-melt polymer films that can be activated and melted during sheet material production by raising the temperature, thus spreading the anti-microbial agents throughout the material when the low-melt films melt and coat the surface of the supporting layer. By varying the amount of anti-microbial-containing low-melt film regionally and/or by varying the amount of anti-microbial agent in these low-melt films, a sheet material can be produced that has a purposely designed regional variation in anti-microbial effectiveness throughout.

Specifically, the latter situation can be achieved by using an amorphous binding film such as PETG, which can be blended to form various types of sheet materials. After heat activation, the PETG melts, wetting the surface of the surrounding films adjacent surface or surfaces. In this way, solidified PETG forms and binds the layers together while spreading the anti-microbial agent throughout the surfaces. Because of the excellent wetting characteristics of PETG, the anti-microbial agent can be uniformly distributed throughout the material. These methods of activating PETG may also be used to additionally distribute other additives described above throughout the finished materials. Other low melt polymers such as Co-Pet, Polyamides, or PE may be used.

It should be understood that the nominal binder or binder component can also be a strength enhancer in some combinations. It will also be understood that other variants including but not limited to combinations, can be made. For example, a first extrusion could produce intermediate film products and such products could be put together with each other or with separate layers.

Another embodiment is a grouping of layers used to practice the invention. One configuration uses PET or other high tenacity polymer at between 20 and 80 percent by weight. Poly 1,4 cyclohexylene dimethylene terephthalate (PCT) or other hydrolysis resistant polymer is used in another layer at a ratio of 80 to 20 percent. One layer is designed to provide the strength and the modulus can be varied to create a high modulus layer, or a low modulus layer, or anywhere in between. The use of PCT in the a layer provides a hydrolysis resistant surface and resistance to long term turbulent fluid flows and chemical wear from the fluids (e.g., soaps present in the fluids.)

One layer made from low temperature polymers with a melting or softening temperature below 200 degrees C., such as PETG, PE, PP, co-PET, or amorphous PET, may be used as binder carrier for anti-microbial additives.

The anti-microbial additives are inorganic compounds of metals such as copper, tin, zinc, silver, etc. The preferred compound is a zeolite (or zirconium phosphate or dissolvable glass) of silver (or copper, zinc, or tin) dispersed in PE, PET, or PBT before being added to the layer. The additives could be added directly to the primary polymer with pre-dispersion. The total active ingredients range from 0.1 to 20 percent by sheet weight.

It will now be apparent to those skilled in the art that other embodiments, improvements, details, and uses can be made consistent with the letter and spirit of the foregoing disclosure and within the scope of this patent, which is limited only by the following claims, construed in accordance with the patent law.

1. An antimicrobial fluid conduit, comprising:
a wall defining a fluid pathway, the wall including a surface upon which is disposed a plastic polymer layer having dispersed therein antimicrobial particles, the plastic polymer layer having a thickness between about 1.73 to 2.5 times the nominal size of the antimicrobial particles so as to substantially constrain the antimicrobial particles to a region at a surface of the plastic polymer layer.

2. The antimicrobial fluid conduit of claim 1, wherein the surface is the inner surface of the wall.

3. The antimicrobial fluid conduit of claim 1, wherein the surface is the outer surface of the wall.

4. The antimicrobial fluid conduit of claim 1, wherein:
the wall is composed of a plastic polymer; and
the wall and the plastic polymer layer having been adhered through co-extrusion.

5. The antimicrobial fluid conduit of claim 1, wherein the wall is composed of one or more polymers selected from the group consisting of thermoformable polymers and thermosetting polymers.

6. The antimicrobial fluid conduit of claim 1, wherein the plastic polymer layer is composed of one or more polymers selected from the group consisting of thermoformable polymers and thermosetting polymers.

7. The antimicrobial fluid conduit of claim 1, wherein the antimicrobial particles include one or more inorganic antimicrobial additives.

8. The antimicrobial fluid conduit of claim 7, wherein the antimicrobial particles comprise a combination of copper and silver particles.

9. The antimicrobial fluid conduit of claim 1, wherein the antimicrobial particles are carried in a carrier selected from the group consisting of zeolites, zirconium phosphate and dissolvable glass.

10. The antimicrobial fluid conduit of claim 1, wherein the wall defines a substantially circular cross-sectional area having a diameter ranging from about 0.1 inches to 18 feet.

11. The antimicrobial fluid conduit of claim 1, wherein in the wall defines a hollow tube.

12. The antimicrobial fluid conduit of claim 1, wherein the wall comprises a pipe.

13. The antimicrobial fluid conduit of claim 1, wherein:
the wall comprises a metallic pipe; and
the plastic layer comprises a subsequently applied film.

14. The antimicrobial fluid conduit of claim 1, wherein the plastic polymer layer further includes additives embedded therein selected from the group consisting of hydrophilic materials, hydrophobic materials, flame retarders, anti-odor additives, and anti-stain materials.
